(19) United States
(12) Patent Application Publication
(10) Pub. No.: US 2013/0101615 A1
(43) Pub. Date: Apr. 25, 2013

(54) METHODS FOR PREVENTING OR TREATING A DISEASE OR CONDITION ASSOCIATED WITH MYCOBACTERIUM AVIUM SUBSPECIES PARATUBERCULOSIS

(71) Applicant: Robert J. Greenstein, Tenafly, NJ (US)

(72) Inventor: Robert J. Greenstein, Tenafly, NJ (US)

(21) Appl. No.: 13/714,716

(22) Filed: Dec. 14, 2012

Related U.S. Application Data
(63) Continuation-in-part of application No. 12/119,657, filed on May 13, 2008.
Application No. 12/119,657 is a continuation-in-part of application No. 11/831,037, filed on Jul. 31, 2007, now abandoned.
Application No. 11/831,037 is a continuation-in-part of application No. PCT/US2006/062316, filed on Dec. 19, 2006.
Application No. PCT/US2006/062316 is a continuation-in-part of application No. 12/956,064, filed on Nov. 30, 2010.
Application No. 12/956,064 is a continuation-in-part of application No. 12/108,144, filed on Apr. 23, 2008, now Pat. No. 7,846,420.
(60) Provisional application No. 60/751,957, filed on Dec. 20, 2005.
(60) Provisional application No. 60/779,541, filed on Mar. 6, 2006.
(60) Provisional application No. 60/745,439, filed on Apr. 24, 2006.
(60) Provisional application No. 60/806,007, filed on Jun. 28, 2006.
(60) Provisional application No. 60/821,734, filed on Aug. 8, 2006.
(60) Provisional application No. 60/822,442, filed on Aug. 15, 2006.
(60) Provisional application No. 60/913,315, filed on Apr. 23, 2007.

(51) Int. Cl. A61K 39/04; A61K 31/606; A61K 31/52; A61K 31/454; A61K 38/13; A61K 31/436; A61K 31/519; A61K 31/635

(52) U.S. Cl. CPC A61K 39/04; A61K 31/519; A61K 31/606; A61K 31/635; A61K 31/454; A61K 38/13; A61K 31/436; A61K 31/52
USPC 424/190.1; 514/249; 514/162; 514/156; 514/323; 514/20.5; 514/291; 514/263.3

(57) ABSTRACT

This invention is a method for immunizing, preventing or treating a Mycobacterium avium subspecies paratuberculosis-associated disease or condition by screening subjects for markers indicative of predisposition or susceptibility to a Mycobacterium avium subspecies paratuberculosis-associated disease or condition and administering a Mycobacterium avium subspecies paratuberculosis vaccine or prophylactic or therapeutic agent.

This application is a continuation-in-part application of U.S. Ser. No. 12/119,657, filed May 13, 2008, which is a continuation-in-part application of U.S. Ser. No. 11/831,037 filed Jul. 31, 2007, now abandoned, which is a continuation-in-part application of PCT/US2006/062316, filed Dec. 19, 2006, which claims benefit of U.S. Provisional Patent Application Ser. Nos. 60/751,957 filed Dec. 20, 2005, 60/779,541 filed Mar. 6, 2006, 60/745,439 filed Apr. 24, 2006, 60/806,007 filed Jun. 28, 2006, 60/821,734 filed Aug. 8, 2006 and 60/822,442 filed Aug. 15, 2006; and a continuation-in-part application of U.S. Ser. No. 12/956,064, filed Nov. 30, 2010, which is a continuation-in-part application of U.S. Ser. No. 12/108,144, filed Apr. 23, 2008, now issued as U.S. Pat. No. 7,846,420 which claims benefit of U.S. Provisional Patent Application Ser. No. 60/913,315, filed Apr. 23, 2007; and the contents of which are incorporated herein by reference in their entireties.

### BACKGROUND OF THE INVENTION

Johne's disease is a chronic diarrheal enteric disease in ruminants that is caused by Mycobacterium avium subspecies paratuberculosis (MAP) (Johne & Frothingham (1895) Dtsch. Zeitschr. Tiermed. Vergl. Pathol. 21:438-454). Live MAP is shed into the milk of cows with Johne's disease (Sweeney (1996) Vet. Clin. North Am. Food Anim. Pract. 12(2):305-12). MAP has been cultured from commercially available pasteurized milk in Europe and the United States (Grant (1998) Appl. Environ. Microbiol. 64(7):2760-1; Ellingson, et al. (2005) J. Food Prot. 68(5):966-72). When Crohn's disease was first described (Crohn, et al. (1932) J. Amer. Med. Assoc. 99:1323-1328), similarities to Johne's disease were identified (Dalziel (1913) Br. Med. J. ii:1068-1070). However, in humans MAP exists in the cell wall-deficient form (Chiodini (1987) J. Clin. Microbiol. 25:796-801). Therefore, in the early analysis of Crohn's disease, MAP could not be detected in humans by the mycobacterial identification techniques of the time, because such techniques stained the mycobacterial cell wall (Ziehl (1882) Dtsch. Med. Wschr. 8:451; Neelsen (1883) Zbl. Med. Wiss. 21:497-501). However, since 1913 the presence of MAP has been identified in humans by other means (see, e.g., Greenstein (2003) Lancet Infect. Dis. 3(8):507-14) and an infectious etiology has been posited for some (Hermon-Taylor (1998) Ital. J. Gastroenterol. Hepatol. 30(6):607-10; Borody, et al. (2002) Dig. Liver Dis. 34(1):29-38), or all (Greenstein (2005) Genetics, Barrier Function, Immunologic & Microbial Pathways. Munster, Germany:25) of inflammatory bowel disease (IBD).

Since the first detection of MAP RNA (Mishina, et al. (1996) Proc. Natl. Acad. Sci. USA 93(18):9816-9820), MAP has been suggested as being the primary and unique, etiological agent of all IBD (Naser, et al. (2004) Lancet 364(9439):1039-1044; Autschbach, et al. (2005) Gut 54(7):944-9; Greenstein (2005) supra; Greenstein (2005) Genetics, Barrier Function, Immunologic & Microbial Pathways. Munster, Germany:24; Greenstein (2005) Crohn's and Colitis Foundation (CCFA) National Research and Clinical Conference. Fourth Annual Advances in Inflammatory Bowel Disease. Miami, Fla.:211) including Perforating and Non-perforating Crohn's disease (Greenstein, et al. (1988) GUT 29:588-592; Gilberts, et al. (1994) Proc. Natl. Acad. Sci. USA 91(126):12721-12724) and ulcerative colitis. It is believed that the particular clinical presentation of IBD that manifests is dependent upon the infected individual's immune response to MAP (Gilberts, et al. (1994) supra). This is analogous to another mycobacterial disease, leprosy. There are two clinical forms of leprosy, tuberculoid and lepromatous (Hansen (1874) Norsk Magazin Laegevidenskaben 4:1-88), both of which are caused by the same organism, M. leprae. The form of leprosy that manifests in a given individual is determined by the immune response of the infected patient (Yamamura, et al. (1991) Science 254:277-279), not by the phenotype or genotype of the leprosy bacillus.

It has been suggested that Koch's postulates (Koch (1882) Berl. Klin. Wschr. 19:221-230), originally promulgated for use in demonstrating tuberculosis infection, may have been met for MAP in Crohn's disease (Greenstein (2003) supra) and more recently for MAP in ulcerative colitis (Greenstein (2005) supra; Naser, et al. (2004) supra).

The link between MAP infection and other diseases is under investigation. An association between ulcerative colitis and Multiple Sclerosis has been suggested (Rang, et al. (1982) The Lancet pg. 555) and the positive association between IBD incidence rates and Multiple Sclerosis has led to the suggestion that these two chronic, immunologically-mediated diseases may have a common environmental etiology (Green, et al. (2006) Am. J. Epidemiol. 164(7):615-23). However, the common causal agent of ulcerative colitis and Multiple Sclerosis was not identified. Moreover, while the symptoms of Multiple Sclerosis have been ameliorated with variety of therapeutic agents including azathioprine, methotrexate, cyclophosphamide and mitoxantrone (Kaffaroni, et al. (2006) Neurol. Sci. 27 Suppl. 1:S13-7), which have been suggested to mediate the secondary inflammatory response, there has been no indication that these agents affect the primary etiological agent.

Sulfasalazine, developed in 1942, has been used to treat rheumatoid arthritis and ulcerative colitis (Svartz (1942) Acta Medica Scandinavica 110:577-598). Sulfasalazine has become, because of empirically observed clinical efficacy, the most common medicine used to treat IBD (Berardi (1997) In: Herfindal E T, Gourley D R, eds. Textbook of Therapeutics. Drugs and Disease Management. Baltimore: Williams and Wilkins, pp. 483-502), with greatest efficacy in ulcerative colitis (Travis (2002) Gut 51(4):548-9; Green, et al. (1998) Aliment. Pharmacol. Ther. 12(12):1207-16; Stenson & Korznik (2003) In: Yamada T, ed. Textbook of Gastroenterology. 4th ed. Lippincott Williams & Wilkins, Philadelphia, Pa., pp. 1727-1828). Sulfasalazine is a conjugate composed of 5-aminosalicylic acid (5-ASA) and sulfapyridine. It is generally accepted that the active moiety of sulfasalazine is 5-ASA (Azad, et al. (1977) Lancet 2:829-31).

5-Aminosalicylic acid (5-ASA, a component of sulfasalazine, olsalazine and balsalazide), and 5-ASA derivatives such as 4-aminosalicylic acid (4-ASA), have been used in the treatment of all forms of IBD, with greatest efficacy in ulcerative colitis (Stenson & Korznik (2003) Textbook of Gastroenterology. Fourth Ed., Lippincott Williams & Wilkins, Philadelphia, Pa. pg. 1727-1828; Schreiber, et al. (1994) Gut 34:1081-1085). 4-aminosalicylic acid, and its derivatives, exhibit tuberculostatic activity (Ertan, et al. (1985) Mikrobiyol. Bul. 19:121-6; Brown & Ratledge (1975) Biochim. Biophys. Acta 385:207-20) and have been suggested for use as adjunct therapeutics to anti-atypical mycobacterial agents such as clarithromycin, rifabutin, and rifampicin (U.S. Pat. No. 6,551,632). Similarly, aspirin (acetylsalicylic acid) and salicylic acid are known to reduce bacterial titers in experimental endocarditis (Nicolau, et al. (1993) Infect. Immun. 61:1593-5; Kupferwasser, et al. (2003) J. Clin. Invest. 112(2):222-33), and 2-(p-aminobenzenesulphonamido) pyridine has antibacterial efficacy in vitro (Whitby (1938) Lancet 1:1210-1212) and has been used as an antibiotic clinically (Evans & Gaisford (1938) Lancet 2:14-19). However, anti-MAP activity of 5-ASA and other salicylic acid derivatives as a primary therapeutic in the treatment of IBD has not been posited or demonstrated. Rather, the effect of 5-ASA in the treatment of IBD has been associated with its “non-specific anti-inflammatory” activity (Stenson & Korznik (2003) supra) with the suggestion that sulfasalazine does not exhibit an antimycobacterial effect in Crohn's disease (Van Caekenberghe (1989) J. Lab. Clin. Med. 114:63-5).

There is increasingly compelling evidence that MAP may be zoonotic (Greenstein & Collins (2004) Lancet 364(9432):396-7) and a human pathogen in gastrointestinal disease (Greenstein (2005) supra) and other diseases as well. There is an additional indication that in man, MAP is systemic and not confined to the gastrointestinal tract (Naser, et al. (2000) Am. J. Gastroenterol. 95(4):1094-5; Naser, et al. (2004) Lancet 364(9439):1039-1044). It is suggested that the reason MAP is zoonotic and has been missed as an etiological agent is that the medical profession has been unknowingly treating MAP with anti-inflammatory agents (e.g., 5-amino salicylic acid, methotrexate, and 6-mercaptopurine), which in fact have anti-MAP activity (Greenstein, et al. (2007) PLoS ONE 2:e161; Greenstein, et al. American Society of Microbiology 2007, Toronto, Canada). It is therefore of concern that viable MAP is found in the food chain (Eltholth, et al. (2009) J. Appl. Microbiol. 107:1061-1071), including pasteurized milk (Ellingson, et al. (2005) supra), and potable chlorinated municipal water (Mishina, et al. (1996) Proc. Natl. Acad. Sci. USA 93:9816-9820).

### SUMMARY OF THE INVENTION

The present invention is a method for preventing or treating a disease or condition associated with a Mycobacterium avium subspecies paratuberculosis (MAP) infection by screening a subject for the presence of one or more markers indicative of predisposition or susceptibility to a disease or condition associated with a MAP infection; selecting a vaccine or prophylactic or therapeutic agent for the subject based upon the presence of the one or more markers; and administering to the subject the vaccine or prophylactic or therapeutic agent to prevent or treat the disease or condition associated with the MAP infection. In one embodiment, the vaccine includes at least one Mycobacterium avium subspecies paratuberculosis (MAP) antigen (e.g., GroES, AhpD, 32 kDa antigen, 34 kDa antigen, 34.5 kDa antigen, 35 kDa antigen, 36 kDa antigen, 42 kDa antigen, 44.3 kDa antigen, 65 kDa antigen or AhpC antigen), or attenuated or killed MAP (e.g., cell wall-competent or cell wall-deficient MAP). In another embodiment, the vaccine further includes at least one antigen isolated from a member of the M. tuberculosis complex (e.g., M. tuberculosis, M. bovis, M. bovis Calmette-Guérin, M. africanum, M. canetti, M. caprae, M. pinnipedii and M. microti), or an attenuated or killed mycobacterium from the MTC. In yet other embodiments, the prophylactic or therapeutic agent comprises one or more of a folate antagonist, a salicylic acid, a purine inhibitor, a thymidylate synthase inhibitor, an antibiotic, an immunosuppressant, a thalidomide, or a prodrug or metabolite thereof.

### DETAILED DESCRIPTION OF THE INVENTION

Epidemiological analysis identifies a parallelism in the increasing incidence of Crohn's disease, ulcerative colitis and Multiple Sclerosis (Green, et al. (2006) supra). In Alzheimer's disease the use of “anti-inflammatories” shows therapeutic benefit (Rogers, et al. (1993) Neurology 43(8):1609-11). Additionally, there is the suggestion that rheumatoid arthritis is protective against Alzheimer's disease (McGeer, et al. (1990) Lancet 335(8696):1037). Analogous to lepromatous leprosy (Hansen (1874) Norsk Magazin for Laegevidenskaben 4:1-88) and tuberculoid leprosy, it is now posited that Multiple Sclerosis and perforating Crohn's disease (Gilberts, et al. (1994) Proc. Natl. Acad. Sci. USA 91(126):12721-12724) are the “acute” forms of a Mycobacterium avium subspecies paratuberculosis (MAP; basonym M. paratuberculosis) infection, whereas Alzheimer's Disease and obstructive Crohn's or ulcerative colitis are the chronic forms of a MAP infection. It is further posited that a causative relationship between MAP and diseases such as IBD and Multiple Sclerosis have been missed because it has not been appreciated that standard “immunomodulatory” treatment regimes, whose mechanisms of actions are unknown or speculated upon, are in fact effective because they are treating a MAP infection. It is posited that MAP is also responsible for a variety of diseases where an infectious etiology has been suggested, e.g., sarcoidosis, ankylosing spondylitis, psoriasis, and psoriatic arthritis and rheumatoid arthritis. Coincidentally, these diseases are often treated with “immunomodulatory” and “anti-inflammatory” agents that have now been shown to interfere with the growth kinetics of MAP.

While some reports have indicated that high-temperature short-time pasteurization does not effectively kill MAP in milk (Grant, et al. (1998) Lett. Appl. Microbiol. 26:166-170; Grant, et al. (1999) Lett. Appl. Microbiol. 28:461-465), killing by turbulent-flow conditions has been demonstrated (Stabel, et al. (1997) Appl. Environ. Microbiol. 63:4975-4977). Given the identification of potential sources of infection and that MAP is widespread over the industrialized as well as non-industrialized world and, a multipronged approach including vaccines, antibiotics, and public health measures are needed to control and prevent MAP infections as well as infections by one or more members of the M. tuberculosis complex (MTC).

Diseases often classified as auto-immune diseases may render the afflicted individual immune-incompetent against MAP. Such an individual would be predisposed or susceptible to a MAP infection, and therefore benefit from MAP vaccination or life-long anti-MAP therapy. Therefore, this invention provides the use genetic or biomolecular profiles to identify subjects who may be predisposed or susceptible to a MAP infection and likely to respond to a MAP vaccine or anti-MAP agent.

Accordingly, this invention provides methods for immunizing, preventing or treating human or non-human subjects for a mycobacterial infection by screening subjects for markers indicative of predisposition or susceptibility to a MAP-associated disease or condition. Upon identification of subjects predisposed to one or more MAP-associated diseases or conditions, and therefore likely to respond to vaccination or treatment, an appropriate therapy is selected for the subjects. Therapies include the administration of a MAP vaccine and/or prophylactic or therapeutic treatment with one or more anti-MAP agents.

As used herein, a MAP-associated disease or condition is a disease or condition having a causative relationship with MAP. This includes diseases and conditions that are caused by a MAP infection and/or exacerbated by a MAP infection. MAP-associated diseases or conditions of this invention include Multiple Sclerosis, Crohn's disease, Alzheimer's Disease, Inflammatory Bowel Disease, ulcerative colitis, rheumatoid arthritis, and in some embodiments also includes sarcoidosis, ankylosing spondylitis, psoriasis and psoriatic arthritis.

Markers indicative of susceptibility or predisposition to a MAP-associated disease or condition include one or more genes, polymorphisms, gene expression levels, polypeptide sequences, or metabolites that have been correlated with the predisposition, presence or propensity to develop a MAP-associated disease or condition. Such markers are known in the art and one or combination of these markers can be used in the screening step of this method.

For example, markers associated with Multiple Sclerosis include, but are not limited to, the 403G/A promoter polymorphism of the Regulated upon Activation, Normal T-cell Expressed and Secreted (RANTES) (Gade-Andavolu, et al. (2004) Mult. Scler. 10:536-9); mtDNA K* haplotype defined by variants at nucleotides 9055, 10,398 and 14,798 (Vyshkina, et al. (2008) Clin. Immunol. 129:31-35); the C-allele of the marker rs391745 near the HERV-Fc1 locus (Hansen, et al. (2011) PLoS One 6:e26438); major histocompatibility complex (MHC) haplotypes, especially those containing HLADRB1*1501 (HLA DRb1 gene, 1501 allele; Barcellos et al. (2006) Hum. Mol. Genet. 15(18):2813-24); IL7R (Lundmark, et al. (2007) Nat. Genet. 39(9):1108-13; Peltonen (2007) N. Engl. J. Med. 357(9):927-9); IL2R (Hafler, et al. (2007) N. Engl. J. Med. 357(9):851-62); EVI5 (Hoppenbrouwers, et al. (2008) Genes Immun. 9(4):334-7); CLEC16A and KIF1B (Aulchenko, et al. (2008) Nat. Genet. 40(12):1402-3); TYK2 and H6PD (Ban, et al. (2009) Eur. J. Hum. Genet. 17(10):1309-13; Alcina, et al. (2010) Eur. J. Hum. Genet. 18(5):618-20).

Association scans in predominantly non-Jewish peoples of Europe Crohn's disease studies have identified 71 susceptibility loci associated with Crohn's disease including coding polymorphisms at NOD2, IL23R, ATG16L1 and an intergenic region on chromosome 5p13 (Barrett, et al. (2008) Nat. Genet. 40:955-962; Duerr, et al. (2006) Science 314:1461-1463; Rioux, et al. (2007) Nat. Genet. 39:596-604; Libioulle, et al. (2007) PLoS Genet. 3:e58; Parkes, et al. (2007) Nat. Genet. 39:830-832; Consortium WTCC (2007) Nature 447:661-678). In Ashkenazi Jews, genome-wide associations were mapped to chromosomes 5q21.1 (rs7705924), 2p15 (rs6545946), 8q21.11 (rs12677663), 10q26.3 (rs10734105), and 11q12.1 (rs11229030), implicating genes, including RPL7, CPAMD8, PRG2, and PRG3 (Kenny, et al. (2012) PLoS Genet. 8:31002559). In addition, linkage over a broad, pericentromeric region on chromosome 16, IBD1, has been identified for Crohn's disease (Hugot, et al. (1996) Nature (London) 379:821-823), and is known to be partially due to the presence of three major coding region polymorphisms in NOD2/CARD15 (Hugot, et al. (2001) Nature 411:599-603; Ogura et al. (2001) Nature 411:603-606). These mutations decrease innate immune responsiveness (Bonen, et al. (2003) Gastroenterology 124:140-146) to muramyl dipeptide (Girardin, et al. (2003) J. Biol. Chem. 278:8869-8872; Inohara, et al. (2003) J. Biol. Chem. 278:5509-5512), a minimally active component of peptidoglycan found in the cell walls of gram-positive and -negative bacteria. Defects in NRAMP1 have also been identified in subjects with Inflammatory Bowel Disease (Kojima, et al. (2001) Tissue Antigens 58(6):379-84). A multidrug resistance 1 (MDR1) Ala893 polymorphism has also been correlated with Inflammatory Bowel Disease (Brant, et al. (2003) Am. J. Hum. Genet. 73:1282-92). A genetic association between RAC1 and ulcerative colitis as well as the SNPs rs10951982 and rs4720672 has also been identified (Muise, et al. (2011) Gastroenterology 141:633-41). Furthermore, a common genotypic basis for ulcerative colitis and Crohn's disease has been shown to include IL-22/23 Th17, adaptive immunity, and barrier pathways (Waterman, et al. (2011) Inflamm. Bowel Dis. 17:1936-42).

Genetic variants in amyloid precursor protein and presenilin 1 and 2 (PSEN1 and PSEN2) have been associated with rare early-onset of Alzheimer's Disease, and apolipoprotein E (APOE) for the common late-onset form (Strittmatter, et al. (1993) Proc. Natl. Acad. Sci. 90:1977-81). Genome-wide association studies have identified nine additional genes/loci for late-onset Alzheimer's Disease, including CR1, BIN1, CLU (a.k.a. APOJ), PICALM, MS4A4/MS4A6E, CD2AP, CD33, EPHA1 and ABCA7 (Lambert, et al. (2009) Nat. Genet. 41:1094-1099; Harold, et al. (2009) Nat. Genet. 41:1088-1093; Seshadri, et al. (2010) JAMA 303:1832-1840; Hollingworth, et al. (2011) Nat. Genet. 43:429-435; Naj, et al. (2011) Nat. Genet. 43:436-441). In addition, meta-analysis of SNPs has revealed an association in the PPP1R3B gene (SNP rs3848140; Kamboh, et al. (2012) Transl. Psychiatry 2:e117). CSF VILIP-1 levels have also been shown to differentiate individuals with Alzheimer's Disease from cognitively normal controls and individuals with other dementias (Tarawneh, et al. (2001) Ann. Neurol. 70:274-285).

Genome-wide association studies have identified shared susceptibility genes for psoriasis and Crohn's disease, including IL23R, IL12B, REL, and TYK2 (Franke, et al. (2010) Nat. Genet. 42:1118-1125; Strange, et al. (2010) Nat. Genet. 42:985-990; Duerr, et al. (2006) Science 314:1461-63; Cargill, et al. (2007) Am. J. Hum. Genet. 80:273-90; Nair, et al. (2008) J. Invest. Dermatol. 128:1653-61; Einarsdottir, et al. (2009) BMC Med. Genet. 10:8). Similarly, seven susceptibility loci, 9p24 near JAK2, 10q22 at ZMIZ1, 11q13 near PRDX5, 16p13 near SOCS1, 17q21 at STAT3, 19p13 near FUT2, and 22q11 at YDJC, are shared between psoriasis and Crohn's disease (Ellinghaus, et al. (2012) Am. J. Hum. Genet. 90:636-647). Linkage to the human leukocyte-associated antigen C gene (HLA C) has been reported in psoriasis and association studies have revealed that the HLA Cw*0602 allele is most commonly associated (Nair, et al. (1997) Hum. Mol. Genet. 6:1349-56; Trembath, et al. (1997) Hum. Mol. Genet. 6:813-20; Burden, et al. (1998) J. Invest. Dermatol. 110:958-60).

A high degree of correlation has been found between rheumatoid arthritis and the presence of antifilaggrin antibody, antikeratin antibody, antibodies to Sa and antibodies to cyclic citrullinated peptide (Goldbach-Mansky, et al. (2000) Arthritis Res. 2:236-43). The presence of rheumatoid factor (RF) has also been widely used as a diagnostic marker for rheumatoid arthritis (Sutton, et al. (2000) Immunol. Today 4:177-183). Furthermore, the serum levels of the soluble form of interleukin (IL)-18 receptor α (IL-18Rα) in rheumatoid arthritis were significantly higher than those in the healthy controls (Takei, et al. (2011) Arthritis Res. Ther. 13:R52). An association with susceptibility and severity of rheumatoid arthritis have been identified for HLA-DRB1, PTPN22, STAT4, OLIG3/TNFAIP3 and TRAF1/C5 (Morgan, et al. (2010) Arthritis Res. Ther. 12:R57).

Using one or more of the above-referenced markers, subjects can be screened in accordance with the present method to identify those who may be predisposed or susceptible to a MAP infection and likely to respond to a MAP vaccine or anti-MAP agent. The detection of genetic and biological markers is routinely practiced in the art and any suitable method can be used.

For example, detection of point mutations or additional base pair repeats can be accomplished by molecular cloning of the specified allele and subsequent sequencing of that allele using techniques known in the art. Alternatively, gene sequences can be amplified directly from a genomic DNA preparation using PCR, and the sequence composition is determined from the amplified product. As described more fully below, numerous methods are available for analyzing a subject's DNA for mutations at a given genetic locus.

One such detection method is allele specific hybridization using probes overlapping the polymorphic site and having about 5, 10, 20, 25, or alternatively 30 nucleotides around the polymorphic region. In another embodiment of the invention, several probes capable of hybridizing specifically to the allelic variant are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips includes oligonucleotides, also termed “DNA probe arrays” is described, e.g., in Cronin, et al. (1996) Human Mutation 7:244.

In other detection methods, it is necessary to first amplify at least a portion of the gene of interest prior to identifying the allelic variant. Amplification can be performed, e.g., by PCR and/or LCR, according to methods known in the art. In one embodiment, genomic DNA of a cell is exposed to two PCR primers and amplification for a number of cycles sufficient to produce the required amount of amplified DNA.

Alternative amplification methods include: self-sustained sequence replication (Guatelli, et al. (1990) Proc. Natl. Acad. Sci. USA 87:1874-1878), transcriptional amplification system (Kwoh, et al. (1989) Proc. Natl. Acad. Sci. USA 86:1173-1177), Q-Beta Replicase (Lizardi, et al. (1988) Bio/Technology 6:1197), or any other nucleic acid amplification method, followed by the detection of the amplified molecules using techniques known to those of skill in the art. These detection schemes are useful for the detection of nucleic acid molecules if such molecules are present in very low numbers.

In one embodiment, any of a variety of sequencing reactions known in the art can be used to directly sequence at least a portion of the gene of interest and detect allelic variants, e.g., mutations, by comparing the sequence of the sample sequence with the corresponding wild-type (control) sequence. Exemplary sequencing reactions include those based on techniques developed by Maxam & Gilbert (1997) Proc. Natl. Acad. Sci. USA 74:560 or Sanger, et al. (1977) Proc. Nat. Acad. Sci. USA 74:5463. It is also contemplated that any of a variety of automated sequencing procedures can be utilized when performing the subject assays, including sequencing by mass spectrometry (see, for example, U.S. Pat. No. 5,547,835; WO 94/16101; U.S. Pat. No. 5,547,835; WO 94/21822; U.S. Pat. No. 5,605,798; Cohen, et al. (1996) Adv. Chromat. 36:127-162; and Griffin, et al. (1993) Appl. Biochem. Bio. 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out. Yet other sequencing methods are disclosed, e.g., in U.S. Pat. No. 5,580,732 and U.S. Pat. No. 5,571,676.

In some cases, the presence of the specific allele in DNA from a subject can be shown by restriction enzyme analysis. For example, the specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (see, e.g., Myers, et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of the allelic variant of the gene of interest with a sample nucleic acid, e.g., RNA or DNA, obtained from a biological sample. The double-stranded duplexes are treated with an agent that cleaves single-stranded regions of the duplex such as duplexes formed based on base-pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, U.S. Pat. No. 6,455,249; Cotton, et al. (1988) Proc. Natl. Acad. Sci. USA 85:4397; Saleeba, et al. (1992) Methods Enzy. 217:286-295. In another embodiment, the control or sample nucleic acid is labeled for detection.

In other embodiments, alterations in electrophoretic mobility are used to identify the particular allelic variant. For example, single-strand conformation polymorphism (SSCP) may be used to detect differences in electrophoretic mobility between mutant and wild-type nucleic acids (Orita, et al. (1989) Proc Natl. Acad. Sci. USA 86:2766; Cotton (1993) Mutat. Res. 285:125-144; Hayashi (1992) Genet. Anal. Tech. Appl. 9:73-79). In another preferred embodiment, the subject method utilizes heteroduplex analysis to separate double stranded heteroduplex molecules on the basis of changes in electrophoretic mobility (Keen, et al. (1991) Trends Genet. 7:5).

In yet another embodiment, the identity of an allelic variant is obtained by analyzing the movement of a nucleic acid comprising the polymorphic region in polyacrylamide gels containing a gradient of denaturant, which is assayed using denaturing gradient gel electrophoresis (DGGE) (Myers, et al. (1985) Nature 313:495).

Examples of techniques for detecting differences of at least one nucleotide between two nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki, et al. (1986) Nature 324:163); Saiki, et al. (1989) Proc. Natl. Acad. Sci. USA 86:6230; Wallace, et al. (1979) Nucl. Acids Res. 6:3543). Such allele specific oligonucleotide hybridization techniques may be used for the detection of the nucleotide changes in the polymorphic region of the gene of interest. For example, oligonucleotides having the nucleotide sequence of the specific allelic variant are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs, et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton, et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini, et al. (1992) Mol. Cell. Probes 6:1).

In another embodiment, identification of the allelic variant is carried out using an oligonucleotide ligation assay (OLA), as described, e.g., in U.S. Pat. No. 4,998,617 and in Landegren, et al. (1988) Science 241:1077-1080. Several techniques based on this OLA method have been developed and can be used to detect the specific allelic variant of the polymorphic region of the gene of interest. For example, U.S. Pat. No. 5,593,826 discloses an OLA using an oligonucleotide having 3′-amino group and a 5′-phosphorylated oligonucleotide to form a conjugate having a phosphoramidate linkage.

The invention further provides methods for detecting the single nucleotide polymorphism in the gene of interest. Because single nucleotide polymorphisms constitute sites of variation flanked by regions of invariant sequence, their analysis requires no more than the determination of the identity of the single nucleotide present at the site of variation and it is unnecessary to determine a complete gene sequence for each patient. Several methods have been developed to facilitate the analysis of such single nucleotide polymorphisms.

In one embodiment, the single base polymorphism can be detected by using a specialized exonuclease-resistant nucleotide, as disclosed, e.g., in U.S. Pat. No. 4,656,127. According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of the polymorphic site (see, e.g., WO 91/02087). As in U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA is described in WO 92/15712. This method uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated.

Several primer-guided nucleotide incorporation procedures for assaying polymorphic sites in DNA have also been described (Komher, et al. (1989) Nucl. Acids. Res. 17:7779-7784; Sokolov (1990) Nucl. Acids Res. 18:3671; Syvanen, et al. (1990) Genomics 8:684-692; Kuppuswamy, et al. (1991) Proc. Natl. Acad. Sci. USA 88:1143-1147; Prezant, et al. (1992) Hum. Mutat. 1:159-164). These methods differ from GBA in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, et al. (1993) Amer. J. Hum. Genet. 52:46-59).

If the polymorphic region is located in the coding region of the gene of interest, yet other methods than those described above can be used for determining the identity of the allelic variant. For example, identification of the allelic variant, which encodes a mutated signal peptide, can be performed by using an antibody specifically recognizing the mutant protein in, e.g., immunohistochemistry or immunoprecipitation. Antibodies to the wild-type or signal peptide mutated forms of the signal peptide proteins can be prepared according to methods known in the art.

In one aspect the invention, a panel of genetic markers is selected from, but not limited to, the genetic markers referenced above. The panel includes probes or primers that can be used to amplify and/or for determining the molecular structure of the markers identified above. The probes or primers can be attached or supported by a solid phase support such as, but not limited to a gene chip or microarray and be provided in a pre-packaged diagnostic kit. The probes or primers can be detectably labeled. This aspect of the invention is a means to identify the genotype of a patient sample for the genes of interest identified above. In one aspect, the methods of the invention provide for a means of using the panel to identify or screen patient samples for the presence of the genetic marker described herein.

A variety of methods can also be used to determine the presence or absence of gene expression. The presence or absence of gene expression may be determined directly or indirectly from the individual's nucleic acid. Analysis of the nucleic acid from an individual, whether amplified or not, may be performed using any of various techniques. For example, the presence or absence of gene expression may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

Other well-known approaches for determining the presence or absence of gene expression include automated sequencing and RNAase mismatch techniques (Winter, et al. (1985) Proc. Natl. Acad. Sci. USA 82:7575-7579). Furthermore, one skilled in the art understands that, where the presence or absence of expression of multiple genes is to be determined, individual gene expression can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that expression of multiple genes can be detected in individual reactions or in a single reaction (a “multiplex” assay).

There are also many techniques readily available in the field for detecting the presence or absence of polypeptides or other biomarkers, including protein microarrays. For example, some of the detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Similarly, there are any number of techniques that may be employed to isolate and/or fractionate markers. For example, a marker may be captured using biospecific capture reagents, such as antibodies, aptamers or antibodies that recognize the marker and modified forms of it. This method could also result in the capture of protein interactors that are bound to the proteins or that are otherwise recognized by antibodies and that, themselves, can be markers. The biospecific capture reagents may also be bound to a solid phase. Then, the captured proteins can be detected by SELDI mass spectrometry or by eluting the proteins from the capture reagent and detecting the eluted proteins by traditional MALDI or by SELDI. One example of SELDI is called “affinity capture mass spectrometry,” or “Surface-Enhanced Affinity Capture” or “SEAC,” which involves the use of probes that have a material on the probe surface that captures analytes through a non-covalent affinity interaction (adsorption) between the material and the analyte. Some examples of mass spectrometers are time-of-flight, magnetic sector, quadrupole filter, ion trap, ion cyclotron resonance, electrostatic sector analyzer and hybrids of these.

Alternatively, for example, the presence of markers such as polypeptides may be detected using traditional immunoassay techniques. Immunoassay requires biospecific capture reagents, such as antibodies, to capture the analytes. The assay may also be designed to specifically distinguish protein and modified forms of protein, which can be done by employing a sandwich assay in which one antibody captures more than one form and second, distinctly labeled antibodies, specifically bind, and provide distinct detection of, the various forms. Antibodies can be produced by immunizing animals with the biomolecules. Traditional immunoassays may also include sandwich immunoassays including ELISA or fluorescence-based immunoassays, as well as other enzyme immunoassays.

Prior to detection, markers may also be fractionated to isolate them from other components in a solution or of blood that may interfere with detection. Fractionation may include fractionation of the desired markers from other biomolecules found in the biological sample using techniques such as chromatography, affinity purification, 1D and 2D mapping, and other methodologies for purification known to those of skill in the art. In one embodiment, a sample is analyzed by means of a biochip. Biochips generally comprise solid substrates and have a generally planar surface, to which a capture reagent (also called an adsorbent or affinity reagent) is attached. Frequently, the surface of a biochip includes a plurality of addressable locations, each of which has the capture reagent bound there.

Sample nucleic acids, proteins, and/or analytes for use in the methods of this invention can be obtained from any biological sample including any cell type or tissue of a subject. For example, a subject's bodily fluid (e.g., blood) can be obtained by known techniques (e.g., venipuncture). Alternatively, nucleic acid tests can be performed on dry samples (e.g., hair or skin). Fetal nucleic acid samples can be obtained from maternal blood as described in WO 91/07660. Alternatively, amniocytes or chorionic villi can be obtained for performing prenatal testing. Screening can also be performed in situ directly upon tissue sections (fixed and/or frozen) of patient tissue obtained from biopsies or resections, such that no nucleic acid purification is necessary. Nucleic acid reagents can be used as probes and/or primers for such in situ procedures (see, for example, Nuovo (1992) “PCR In Situ Hybridization Protocols And Applications”, Raven Press, NY).

The identification of subjects predisposed or susceptible to one or more MAP-associated diseases or conditions indicates that the subject is likely to respond to vaccination or treatment. A “response” implies any kind of improvement or positive response either clinical or non-clinical such as, but not limited to, measurable reduction in disease or disease progression, complete response, partial response, stable disease, increase or elongation of progression free survival, increase or elongation of overall survival, or delay in onset. The term “likely to respond” shall mean that the patient is more likely than not to exhibit a response to a MAP vaccine or anti-MAP agent.

In accordance with the methods herein, the selection of an appropriate therapeutic strategy is subsequently determined for subjects predisposed or susceptible to one or more MAP-associated diseases or conditions. Therapies include the administration of a MAP vaccine and/or prophylactic or therapeutic treatment with one or more anti-MAP agents.

For the purposes of the present invention, a MAP vaccine is intended to include whole MAP cells, either cell wall-competent or cell wall-deficient; MAP cell extracts; isolated protein (i.e., a subunit vaccine); or combinations thereof. Whole cell vaccines can be produced from cell well-competent and/or cell wall-deficient MAP which has been inactivated or attenuated or has been killed. Live attenuated vaccines have the advantage of mimicking the natural infection enough to trigger an immune response similar to the response to the wild-type organism. Such vaccines generally provide a high level of protection, especially if administered by a natural route, and some may only require one dose to confer immunity. Because MAP exists in humans in the cell wall-deficient state, a vaccine which targets this obligate intracellular form is desirable. By way of illustration, cell wall-competent and cell wall-deficient (i.e., spheroplasts) vaccine preparations have been shown to reduce lesion scores associated with Johne's Disease in baby goats (Hines, et al. (2005) 8^{th} International Colloquium on Paratuberculosis, Copenhagen, Denmark). MAP can be attenuated using any conventional strategy employed in producing an attenuated M. tuberculosis. For example, serial passage or culture of the active organism in culture media or cells can be employed to attenuate MAP. Alternatively, the vaccine of the present invention can contain heat-killed MAP cells. In this regard, vaccination of calves with a heat-killed field strain of MAP results in high concentrations of IFN-γ and better protection against a MAP challenge exposure than does a commercially available vaccine (Uzonna, et al. (2002) Proc. 7^{th} Intl. Coll. Paratuberculosis; Juste (ed)).

In addition, or as an alternative to an attenuated or killed MAP vaccine, a MAP vaccine can be a MAP subunit vaccine. Any one of the well-known MAP-specific antigens, or antigen fragments thereof, commonly employed in veterinary medicine can be used as a vaccine in accordance with the present invention. See Table 1.

TABLE 1

| | | Size |
|---|---|---|
| MAP protein | Characteristic | (kDa) |
| GroES | Heat shock protein | 10 |
| AhpD | Alkyl hydroperoxide reductase D | 19 |
| 32-kDa antigen | Fibronectin binding properties, | 32 |
| | secreted | |
| 34-kDa antigen | Cell wall antigen, B-cell epitope | 34 |
| 34-kDa antigen | Serine protease | 34 |
| 34.5-kDa antigen | Cytoplasmic protein | 34.5 |
| 35-kDa antigen | Immunodominant protein | 35 |
| 36-kDa antigen | p36 antigen | 36 |
| 42-kDa antigen | Cytoplasmic protein | 42 |
| 44.3-kDa antigen | Soluble protein | 44.3 |
| AhpC | Alkyl hydroperoxide reductase C | 45 |
| 65-kDa antigen | GroEL heat shock protein | 65 |

The 32-kDa secreted protein with fibronectin binding properties has been implicated in protective immunity (Andersen, et al. (1991) Infect. Immun. 59:1905-1910; El-Zaatari, et al. (1994) Curr. Microbiol. 29:177-184) and the 34-kDa cell wall antigenic protein is homologous to a similar immunogenic protein in M. leprae (De Kesel, et al. (1992) Scand. J. Immunol. 36:201-212; De Kesel, et al. (1993) J. Clin. Microbiol. 31:947-954; Gilot, et al. (1993) J. Bacteriol. 175:4930-4935; Silbaq, et al. (1998) Infect. Immun. 66:5576-5579). The seroreactive 34-kDa serine protease expressed in vivo by MAP has also been described (Cameron, et al. (1994) Microbiology 140:1977-1982; however, this antigen is different from the 34-kDa antigen described above. Another strongly immunoreactive protein of kDa has also been identified in M. avium complex isolates, including MAP (El-Zaatari, et al. (1997) J. Clin. Microbiol. 35:1794-1799). A more thoroughly characterized protein of 65 kDa from MAP is a member of the GroEL family of heat shock proteins (El-Zaatari, et al. (1994) Curr. Microbiol. 29:177-184; El-Zaatari, et al. (1995) Clin. Diagn. Lab. Immunol. 2:657-664). Like the GroES proteins, the GroEL antigens from other mycobacteria are highly immunogenic (Shinnick (1987) J. Bacteriol. 169:1080-1088; Thole, et al. (1987) Infect. Immun. 55:1466-1475; Thole, et al. (1988) Infect. Immun. 56:1633-1640).

The alkyl hydroperoxide reductases C and D (AhpC and AhpD) have also been characterized as immunogenic proteins of MAP (Olsen, et al. (2000) Infect. Immun. 68:801-808). Unlike other mycobacteria, large amounts of these antigens are produced by MAP when the bacilli are grown without exposure to oxidative stress. AhpC is the larger of the two proteins and appears to exist as a homodimer in its native form since it migrates at both 45 and 24 kDa under denaturing conditions. In contrast, AhpD is a smaller monomer, with a molecular mass of about 19 kDa. Antiserum from rabbits immunized against AhpC and AhpD reacted only with MAP proteins and not with proteins from other mycobacterial species, indicating that antibodies against these proteins are not cross-reactive. Furthermore, peripheral blood monocytes from goats experimentally infected with MAP were capable of inducing gamma interferon (IFN-γ) responses after stimulation with AhpC and AhpD, confirming their immunogenicity (Olsen, et al. (2000) Infect. Immun. 68:801-808).

In addition to MAP, the vaccine of this invention can also include vaccination against mycobacteria that cause human and/or animal tuberculosis (TB), e.g., those grouped together within the Mycobacterium tuberculosis complex. Members of the M. tuberculosis complex include M. tuberculosis, M. bovis, M. bovis Calmette-Guérin (BCG), M. africanum, M. canetti, M. caprae, M. pinnipedii and M. microti (Huard, et al. (2006) J. Bacteriol. 188:4271-4287). In this respect, the methods of this invention are extended to the prevention and/or treatment of a MAP infection and M. tuberculosis and/or M. bovis infection. Similar to MAP, vaccines against the Mycobacterium tuberculosis complex can include an attenuated or killed mycobacterial vaccine, or a subunit vaccine.

Antigenic proteins of M. tuberculosis are known in the art and include but are not limited to, PirG protein encoded by the Mtb gene Rv3810; PE-PGRS protein encoded by the Mtb gene Rv3367; PTRP protein encoded by the Mtb gene Rv0538; MtrA protein encoded by the Mtb gene Rv3246c; MTb81, Mo2, FL TbH4, HTCC#1 (Mtb40), TbH9, MTCC#2 (Mtb41), DPEP, DPPD, TbRa35, TbRa12, MTb59, MTb82, Erd14 (Mtb16), DPV (Mtb8.4), MSL (Mtb9.8), MTI (Mtb9.9A, also known as MTI-A), Ag85B, ESAT-6, and α-crystalline antigens of M. tuberculosis. In some embodiments, the antigenic protein provides cross-protection against M. tuberculosis and M. bovis, i.e., antibodies to said protein recognize the protein from both M. tuberculosis and M. bovis. In other embodiments, the antigenic protein is specific for M. tuberculosis and absent from the genome of BCG. Examples of such antigens include M. tuberculosis BCG Negative polypeptides, MTBN1-MTBN8. These and other antigens are described, for example, in U.S. Pat. Nos. 7,745,141; 7,579,141; and 7,311,922, incorporated herein by reference.

Antigenic proteins of M. bovis are also known in the art. For example, U.S. Pat. No. 7,670,609 describes a recombinant Bacille Calmette-Guerin (BCG) subunit-based vaccine.

Antigenic proteins disclosed herein, can be prepared and isolated by any conventional method including recombinant production. The term isolated does not require absolute purity; rather, it is intended as a relative definition, and can include preparations that are highly purified or preparations that are only partially purified. When recombinantly produced, antigenic proteins of the invention can also be produced as fusion proteins containing more than one antigen (e.g., fusion of antigen 85B (Ag85B) and ESAT-6) or fusion proteins containing an antigen in combination with an adjuvant or carrier protein.

It is contemplated that various combinations of antigen proteins, and/or attenuated and/or killed mycobacteria can be employed. By way of illustration, a vaccine of the invention can be composed of heat-killed MAP in combination with attenuated BCG. As another example, a vaccine of the invention can include an antigenic protein from MAP in combination with attenuated BCG and proteins Ag85B and ESAT-6 from M. tuberculosis.

Vaccines of the present invention are prepared using routine methods. Generally, vaccines are prepared as injectables, in the form of aqueous solutions or suspensions. Vaccines in an oil base are also well-known such as for inhalation. Solid forms which are dissolved or suspended prior to use can also be formulated. Suitable carriers, diluents and excipients are generally added that are compatible with the active ingredients and acceptable for use in humans and non-human animals. Examples of such carriers include, but are not limited to, water, saline solutions, dextrose, or glycerol. Carriers can also include liposomes or microspheres. Combinations of carriers can also be used. For example, prime immunization with BCG and a subunit vaccine (proteins Ag85B and ESAT-6) in liposomes followed by boosting with the subunit vaccine in conventional adjuvant has been shown to result in an increase in the protective efficacy of up to 7-fold compared with BCG alone and 3-fold compared with unaugmented BCG boosted by subunit vaccine (Dietrich, et al. (2007) J. Immunol. 178:7321-3730). A generally recognized compendium of methods and ingredients of vaccine compositions is Remington: The Science and Practice of Pharmacy, Alfonso R. Gennaro, editor, 20th ed. Lippincott Williams & Wilkins: Philadelphia, Pa., 2000.

Vaccine compositions can further incorporate additional substances to stabilize pH, or to function as adjuvants, wetting agents, or emulsifying agents, which can serve to improve the effectiveness of the vaccine. Examples of suitable adjuvants include, but are not limited to, aluminum salts; Incomplete Freund's adjuvant; threonyl and n-butyl derivatives of muramyl dipeptide; lipophilic derivatives of muramyl tripeptide; monophosphoryl lipid A; 3′-de-O-acetylated monophosphoryl lipid A; cholera toxin; QS21; phosphorothionated oligodeoxynucleotides with CpG motifs and adjuvants disclosed in U.S. Pat. No. 6,558,670.

Vaccines are generally formulated for parenteral administration and are injected either subcutaneously or intramuscularly. Vaccines can also be formulated as suppositories or for oral or nasal administration using methods known in the art. For example,

The amount of vaccine sufficient to confer immunity to pathogenic mycobacteria is determined by methods well-known to those skilled in the art. This quantity will be determined based upon the characteristics of the vaccine recipient and the level of immunity required. Typically, the amount of vaccine to be administered will be determined based upon the judgment of a skilled physician or veterinarian. Where vaccines are administered by subcutaneous or intramuscular injection, a range of 0.5 to 500 μg purified protein can be given.

The present invention is also directed to a vaccine in which an antigenic protein, or antigenic fragment thereof, is delivered or administered in the form of a polynucleotide encoding the protein or fragment (i.e., a DNA vaccine). In DNA vaccination, the subject is administered a polynucleotide encoding a antigenic protein that is then transcribed, translated and expressed in some form to produce strong, long-lived humoral and cell-mediated immune responses to the antigen. The polynucleotide can be administered using viral vectors or other vectors, such as liposomes, and can be combined with an acceptable carrier.

In accordance with the prophylactic or therapeutic treatment of a MAP-associated disease or condition, a subject is administered one or more anti-MAP agents. In particular embodiments, one or a combination anti-MAP agents are administered to inhibit or suppressing the growth of MAP thereby preventing treating diseases or conditions associated with MAP (i.e., MAP is the etiological agent of the disease or condition).

Agents disclosed herein are particularly useful for their bactericidal or bacteriostatic activity, i.e., the degree of efficacy of an antibacterial agent. Antimicrobial compositions can effect two kinds of microbial cell damage. The first is a lethal, irreversible action resulting in complete microbial cell destruction or incapacitation as observed by a decrease in cell proliferation and/or the number of viable cells. The second type of cell damage is reversible, such that if the organism is rendered free of the agent, it can again multiply. The former is termed bactericidal and the later, bacteriostatic.

MAP has a long division time and, akin to the treatment of Mycobacterium tuberculosis and M. leprae, antimicrobial therapy for the treatment of MAP has to be carried out for a long period of time with a risk of the generating resistant strains. Thus, while some embodiments embrace the use of one anti-MAP agent as the primary prophylactic or therapeutic agent, other embodiments embrace the simultaneous use of multiple anti-MAP agents to prevent the emergence of resistant mycobacterium in the prevention or treatment of a MAP infection. Thus, in particular embodiments, the present invention provides the combination of at least two, three, four, five, six, seven or more anti-MAP agents disclosed herein.

When employing a single anti-MAP agent, certain embodiments embrace the use of a salicylic acid or prodrug thereof as the primary or sole therapeutic to inhibit the proliferation of MAP. Salicylic acids of use in accordance with the this invention include, 5-ASA and derivatives thereof, e.g., 4-ASA (also known as p-aminosalicylic acid), NCI60-676931, CAS 69727-10-2,3-aminosalicylic acid, CAS 4136-97-4, CAS 2374-03-0, acetylsalicylic acid, 5-bromo-2-acetylsalicylic acid, N-acetyl-5-aminosalicylic acid; prodrugs thereof, e.g., 5-aminosalicyl-L-aspartic acid and 5-aminosalicyl-L-glutamic acid (see Jung, et al. (2001) J. Pharm. Sci. 90(11):1767-75); and pharmaceutically acceptable salts thereof, e.g., potassium acetylsalicyclic acid (see, U.S. RE38,576). As used in the context of the present invention, a prodrug is an active compound that is modified by, e.g., conjugation with an inactive compound, to facilitate delivery or decrease toxicity of the active compound until it reaches its target site where it undergoes biotransformation via a metabolic process before exhibiting its pharmacological effects. Thus, in one embodiment, the salicylic acid of the instant invention is an aminosalicylic acid or prodrug thereof. In another embodiment, the salicylic acid is 5-ASA or a prodrug thereof.

In certain embodiments, this invention embraces the administration of a folate antagonist either alone or in combination with a salicylic acid, wherein said salicylic acid and folate antagonist are not conjugated at the time of administration. In other embodiments, the salicylic acid and folate antagonist are not conjugated at the site of action.

As used in the context of this invention, a folate antagonist is an agent that inhibits one or more enzymes of the folate biosynthetic pathway. For the purposes of this invention, the term folate antagonist expressly includes folate antagonist prodrugs and folate antagonist metabolites. A metabolite is a product of a parent compound which undergoes biotransformation via a metabolic process; however, the parent compound may also exhibit a pharmacological effect. Sulfonamides are a well-known group of folate antagonists that are mostly derivatives of sulfanilamide (p-aminobenzenesulfonamide). Organisms which synthesize their own folic acid and which cannot use an exogenous supply of the vitamin are sensitive to sulfonamides. This is a result of the ability of sulfonamides to act as structural analogs of p-aminobenzoic acid (PABA) and competitively inhibit the incorporation of PABA during folic acid synthesis. For example, methotrexate is a folate antagonist that competitively and reversibly inhibits DHFR and has been used in the treatment Crohn's disease (Feagan, et al. (2000)N. Engl. J. Med. 342:1627-32). Similarly, it is posited that sulfapyridine, a sulfonamide cleavage product of the IBD therapeutic salfasalazine, also functions as a folate antagonist which inhibits the proliferation of MAP.

There are many sulfonamide drugs that differ in their clinical properties and toxicities. Most are derivatives bearing substituents at the nitrogen of the sulfonamide group (i.e., NH₂C₆H₄SO₂NHR, wherein R represents the substituent). Substitution at the p-amino group normally results in loss of antibacterial activity. However, such derivatives are often hydrolyzed in vivo to an active form and can therefore be administered in an inactive form. For example, p-N-succinylsulfathiazole and phthalylsulfathiazole are inactive but are hydrolyzed in the lower intestine to release the active component sulfathiazole.

Exemplary folate antagonists include sulfonamides such as sulfanilamide; sulfaphenazole; sulfadiazine; sulfamethizole; sulfisoxazole; sulfamethoxazole (4-amino-N-(5-methyl-1,3,4-thiadiazol-2-yl)benzenesulfonamide); sulfapyridine; sulfadimethoxine (4-amino-N-(2,6-dimethoxy-4-pyrimidinyl)benzenesulfonamide); sulfamonomethoxine; sulfadimidine; sulfamethazine; sulfacetamide (N-[4-aminophenyl)sulfonyl]-acetamide); sulfathiazole (4-azino-N-2-thiaamide); sulfaguanidine (4-amino-N-(aminoiminomethyl)benzenesulfonamide).

Additional folate antagonists of use in the instant methods include, but are not limited to, aminopterin, methotrexate, trimetrexate, LY231514, and 5-phosphoribosylglycinamide formyltransferase (GARFT) inhibitors such as lometrexol. Moreover, pyridopyrimidines that inhibit the activity of dihydrofolate reductase (DHFR) from Pneumocystis carinii, Toxoplasma gondii, and Mycobacterium avium (Chan, et al. (2005) J. Med. Chem. 48:4420-31), are also embraced by this invention. In one embodiment, the folate antagonist is a sulfonamide. In other embodiment, the folate antagonist is sulfapyridine.

As indicated, some embodiments embrace a salicylic acid used alone or in combination with a folate antagonist. However, other embodiments of this invention provide the use of one, two, three, four or more anti-MAP agents selected from the group consisting of a folate antagonist, a purine inhibitor, a thymidylate synthase inhibitor, an antibiotic, an immunosuppressant and a thalidomide. In particular embodiments, at least two anti-MAP agents are employed.

During the de novo biosynthesis of purines and thymine, methyl groups are donated by tetrahydrofolate, which is regenerated from the dihydrofolate product of thymidylate synthase by the action of DHFR. Thus, folate antagonists of DHFR indirectly inhibit the biosynthesis of purines and thymine. The 6-mercaptopurine metabolite of azathioprine, an IBD therapeutic, is a purine analogue that acts by directly interfering with purine synthesis. Accordingly, the use of agents that indirectly interfere with purine biosynthesis are included within the scope of anti-MAP agents of this invention. Such inhibitors include purine inhibitors such as 6-mercaptopurine, azathioprine, 6-thioguanine, and 6-methylmercaptopurine riboside (see U.S. Pat. No. 6,355,623) and their prodrugs and metabolites; and thymidylate synthase inhibitors such as raltitrexed (Tomudex), nolatrexed, LY231514, ZD9331, and 5-fluorouracil and its prodrugs, 5′-deoxy-5-fluorouridine and ftorafur, which are metabolized to fluorodeoxyuridine monophosphate. In particular embodiments, the purine inhibitor 6-mercaptopurine is used as an anti-MAP agent.'

The structure of thalidomide is similar to that of the DNA purine bases adenine (A) and guanine (G).

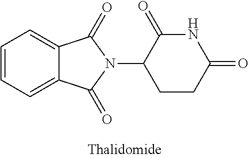 [SMILES: O=C1CCC(N2C(=O)c3ccccc3C2=O)C(=O)N1]

In solution, thalidomide binds more readily to guanine than to adenine, and has almost no affinity for the other nucleotides, cytosine (C) and thymine (T). It is noteworthy that the progenitor molecule of these two purine double ringed nucleotides is the double ringed dihydrofolate that is converted to folic acid prior to becoming the purine nucleotides. There is thus a parallelism between this and the posited action of methotrexate, a dihydrofolate reductase inhibitor which interferes with the DNA replication of the infecting mycobacterium. As thalidomide can intercalate into DNA at G-rich sites (Stephens, et al. (2000) Biochem Pharmacol. 59(12):1489-99) and the 1H-isoindole-1,3(2H)-dione thalidomide metabolite may bind to replication/transcription machinery given the structural similarity to purines (Scheme 1), it is contemplated that thalidomide, or active metabolites thereof, exerts its anti-mycobacterial effect by disrupting prokaryotic DNA replication.

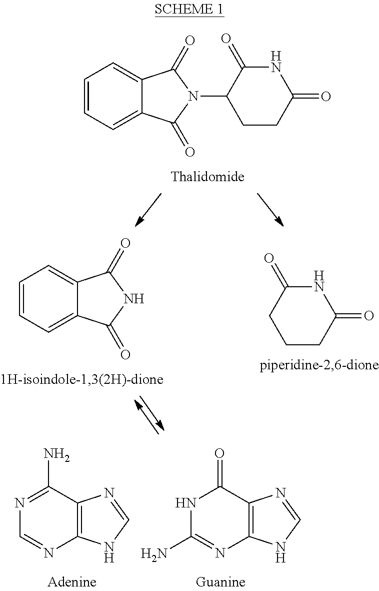 [SMILES: O=C1CCC(N2C(=O)c3ccccc3C2=O)C(=O)N1 ; O=C1NC(=O)c2ccccc21 ; O=C1CCCC(=O)N1 ; Nc1ncnc2[nH]cnc12 ; Nc1nc2[nH]cnc2c(=O)[nH]1]

In this regard, the piperidine metabolite of thalidomide is the basis for a well-known class of antimicrobial agents, namely glutarimides such as streptimidone, streptovitacins and inactone (see, e.g., Kim, et al. (1999) J. Agric. Food Chem. 47(8):3372-80; Sugawara, et al. (1992) J. Antibiot. (Tokyo) 45(9):1433-41; Sonoda, et al. (1991) J. Antibiot (Tokyo) 44(2):160-3), and it has now been demonstrated that 1-hydroxy-2,6-piperidinedione (HPD) inhibits the growth of MAP. Thus, certain embodiments of this invention embrace the use of either the isoindole metabolite or piperidine metabolite alone or, as has been indicated for the breakdown products of sulfasalazine (i.e., sulfapyridine and 5-ASA), in combination to act synergistically in the inhibition of the proliferation of multiple Mycobacterium species and subspecies. Such synergy between the two metabolites of thalidomide can be assessed in vivo in a clinical study, e.g., in either animals or humans, where the intact thalidomide molecule, or its components individually and in combination are prospectively compared.

Moreover, it is posited that the metabolites of thalidomide analogs such as lenalidomide and CC-4047, differing by substituents of the isoindole, will be more potent antimicrobial agents than the metabolites of thalidomide itself as these analogs are at least 2000 times more potent than thalidomide in inhibiting the TNF-α (Teo, et al. (2005) Drug Discovery Today 10:107-114).

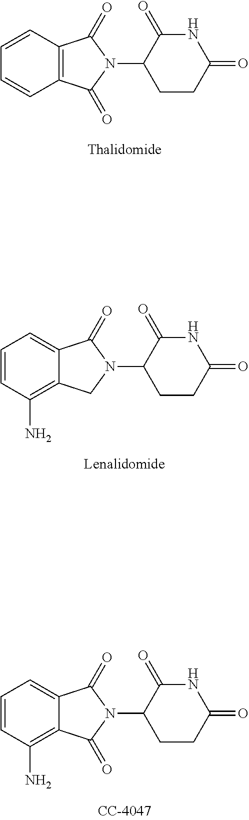 [SMILES: O=C1CCC(N2C(=O)c3ccccc3C2=O)C(=O)N1 ; Nc1cccc2c1CN(C1CCC(=O)NC1=O)C2=O ; Nc1cccc2c1C(=O)N(C1CCC(=O)NC1=O)C2=O]

Thus, in accordance with a particular embodiment of the invention, a thalidomide is used in the methods of the invention. As used herein, the term thalidomide is intended to include, but is not limited to, thalidomide, a derivative or analog of thalidomide, or a pharmaceutically acceptable prodrug (i.e., a compound which is metabolized to produce thalidomide or analogues or biologically active salt forms thereof), a metabolite, salt, solvate, hydrate, or clathrate thereof. Thalidomide contains a chiral center, and is commercially available as a racemate. The methods and compositions of the invention therefore encompass the use of racemic thalidomide as well as optically pure enantiomers of thalidomide. Optically pure enantiomers of thalidomide can be prepared by methods well-known in the art. These include, but are not limited to, resolution of chiral salts, asymmetric synthesis, or chiral chromatography. It is further contemplated that pharmaceutically acceptable prodrugs, salts, solvate, clathrates and derivatives of thalidomide be used in the methods and compositions of the invention. Examples of derivatives of thalidomide that can be used in the methods and compositions of the invention include, but are not limited to, taglutimide, supidimide, and those disclosed in WO 94/20085. Other derivatives of thalidomide encompassed by this invention include, but are not limited to, 6-alkyl-2-[3′-nitrophthalimido]-glutarimide, 6-alkyl-2-[4′-nitrophthalimido]-glutarimide, 6-alkyl-3-phenylglutarimides, CPS11, CPS49, IMiD3, lenalidomide and CC-4047. See, e.g., De and Pal (1975) J. Pharm. Sci. 64(2):262-266 and U.S. Pat. No. 6,235,756. Moreover, 5-OH-thalidomide and 5′-OH-thalidomide metabolites, which exhibit biological activity in vitro (Price, et al. (2002) Ther. Drug Monit. 24(1):104-10), are also contemplated for use in the instant methods, as are 4-OH-thalidomide and N—OH-thalidomide. In some embodiments, the thalidomide itself exhibits activity, whereas in other embodiments, one or more metabolites of the thalidomide exhibit activity (see Scheme 1).

With respect to embodiments drawn to metabolites of thalidomide, e.g., the isoindole metabolite or piperidine metabolite, this invention also embraces a derivative or analog of a thalidomide metabolite, or a pharmaceutically acceptable prodrug, salt, solvate, hydrate, or clathrate thereof. By way of illustration, piperidine-2,6-dione analogues of particular use include, but are not limited to, aminoglutethimide, p-nitro-glutethimide, p-nitro-5-aminoglutethimide, cyclohexy-laminoglutethimide, phenglutarimide, acetylamino-glutethimide, glutethimide, pyridoglutethimide, 3-phenylacetyl-amino-2,6-piperidinedione (antineoplaston A-10), and thioglutethimide.

Another embodiment of the invention includes the use of an anti-MAP antibiotic, or a prodrug or metabolites thereof. For example, macrolide antibiotics are known for use in the treatment of MAP. Macrolide antibiotics of the invention include clarithromycin and azithromycin. Other anti-MAP antibiotics of use in conjunction with the anti-MAP agents identified herein include, but are not limited to, rifabutin, amikacin, cyprofloxicillin, clofazimine, rifampicin, azithromycin, roxithromycin, ethambutol, ofloxacin, ciprofloxacin, metroniadiazole and oxazolidinone.

Having demonstrated the anti-MAP activity of immunosuppressants, particular embodiments of this invention further include the use of an immunosuppressant, either alone or in combination with one or more additional anti-MAP agents for inhibiting the proliferation of MAP and preventing or treating a MAP infection. An immunosuppressant is defined herein as an agent that can suppress or prevent the immune response. In particular embodiments of the invention, the immunosuppressant employed is a cyclosporin or macrolide immunosuppressant. A cyclosporin (also referred to as cyclosporine) is defined herein as a cyclic nonribosomal peptide containing one or more D-amino acids. Cyclosporins of use in accordance with the present invention include, but are not limited to Cyclosporine A, Cyclosporin G (OG37-325), and O-hydroxyethyl-D(Ser)8-cyclosporine (SDZ IMM 125).

A macrolide immunosuppressant is defined herein as a class of compounds sharing a macrolide-like structure composed of a large macrocyclic lactone ring to which one or more deoxy sugars, usually cladinose and desosamine, may be attached. In general, the lactone rings of macrolide immunosuppressants are 14, 15 or 16-membered. Exemplary macrolide immunosuppressants of the invention include, but are not limited to sirolimus (rapamycin), tacrolimus (FK506), pimecrolimus, and everolimus.

As indicated herein, it is posited that the metabolic products of sulfasalazine, namely sulfapyridine and 5-ASA, both exhibit antimicrobial activity. Accordingly, particular embodiments further embrace combining the one, two, three or more anti-MAP agents selected from the group consisting of a folate antagonist, a purine inhibitor, a thymidylate synthase inhibitor, an antibiotic, an immunosuppressant and a thalidomide with a salicylic acid.

When used therapeutically, the one or more anti-MAP agents of the invention are usually administered in the form of an anti-MAP pharmaceutical composition, wherein the agents are in admixture with a pharmaceutically acceptable carrier. Such a pharmaceutical composition can be prepared in any manner well-known in the pharmaceutical art. See, e.g., Remington: The Science and Practice of Pharmacy, Alfonso R. Gennaro, editor, 20th ed. Lippincott Williams & Wilkins: Philadelphia, Pa., 2000. For example, in making the pharmaceutical composition of the invention, the anti-MAP agents are usually mixed with a carrier, diluted by a carrier or enclosed within such a carrier which can be in the form of a capsule, sachet, paper or other container. When the carrier serves as a diluent, it can be a solid, semi-solid, or liquid material, which acts as a vehicle, carrier or medium for the active ingredient. Thus, the pharmaceutical compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In this regard, the anti-MAP composition can be administered by a variety of routes including oral, rectal, transdermal, subcutaneous, intravenous, intramuscular, intranasal or locally, e.g., intraspinal, intracerebral, and the like.

Suitable carriers for use in accordance with the invention include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methylcellulose. The formulations can additionally include lubricating agents such as talc, magnesium stearate, and mineral oil; wetting agents; emulsifying and suspending agents; preserving agents such as methyl- and propylhydroxybenzoates; sweetening agents; and flavoring agents. The pharmaceutical compositions of the invention can be formulated so as to provide quick, sustained or delayed release of the active ingredient after administration to the subject by employing procedures known in the art.

In particular embodiments, the pharmaceutical composition is enterically coated. For example, tablets, capsules or pills can be coated or otherwise compounded to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac and cellulose acetate.

In preventing or treating a MAP infection of the CNS it may be desirable that the one or more anti-MAP agents be attached to a delivery moiety or entrapped within a drug carrier (e.g., liposomes and nanoparticles), which facilitates delivery across the blood-brain barrier. See, e.g., Koziara, et al. (2006) J. Nanosci. Nanotechnol. 6(9-10):2712-35; and de Boer and Gaillard (Sep. 8, 2006) Annu Rev Pharmacol Toxicol. [Epub ahead of print] PMID: 16961459.

For the purposes of this invention, the anti-MAP agents of the invention can be administered simultaneously or sequentially (one immediately following the other) with the proviso that when a folate antagonist is used in combination with a salicylic acid, the folate antagonist and salicylic acid are not conjugated.

As discussed herein, it is believed that the causative relationship between MAP and diseases such as IBD, Multiple Sclerosis and Alzheimer's Disease, as well as other infectious agents in other inflammatory diseases, has been missed because it has not been appreciated that standard inflammatory disease treatment regimes are actually treating the infectious entity. For example, it is believed that the mechanism of action of the individual components of sulfapyridine is to retard the growth of MAP in IBD. Likewise, it is contemplated that the anti-metabolites disclosed herein (i.e., folate antagonists such as methotrexate, purine inhibitors such as 6-mercaptopurine and thymidylate synthase inhibitors) exhibit antibiotic activity in addition to their anti-neoplastic activity. It is believed that anti-metabolites interfere with common metabolic pathways in both eukaryotes and prokaryotes; however because prokaryotes are more sensitive to these agents, much lower doses can be achieved in the treatment of inflammatory diseases resulting from bacterial infections as compared to doses employed in the treatment of human malignancies.

For example, methotrexate and 6-mercaptopurine are used in the therapy of malignancies as well as inflammatory diseases. Remarkably, these agents are employed at different doses; an anti-neoplastic dose and an anti-inflammatory dose.

The anti-metabolite 6-mercaptopurine and its metabolite azathioprine are used to treat two generic types of disease, malignancies and inflammatory diseases (Calabresi & Parks (1977) In: Goodman L S, Gilman A, eds. The Pharmacological Basis of Therapeutics. Fifth ed. New York: Macmillan, pg. 1254-1307). The mechanism of action is “to inhibit nucleotide biosynthesis and purine nucleotide interconversion.” (Berardi (1996) In: Herfindal E T, Gourley D R, eds. Textbook of Therapeutics. Drugs and Disease Management. Baltimore: Williams and Wilkins, pg. 483-502).

“High dose” treatment is used in reticuloendothelial malignancies such as acute leukemia. In such cases the starting dose is 100-200 mg/day 6-mercaptopurine titrated to a maintenance dose of 50-100 mg/day (Calabresi & Parks (1977) supra; Calabresi & Chabner (1990) In: S. GL, Gilman A, Rall T, Nies A S, Taylor P, eds. The Pharmacological Basis of Therapeutics. 8 ed. New York: Pergamon Press, pg. 1202-1208). The cumulative dose that is used, until bone marrow depression occurs, in non-hematological malignancies is ˜45 mg/kg (Range 18-106 mg/kg). Thus, a 70 kg man might receive an acute cumulative dose of 6-mercaptopurine of >7 grams.

In contrast, “low dose” long-term 6-mercaptopurine is used in the therapy of inflammatory diseases such as IBD and rheumatoid arthritis at a dose that is at ˜≦25% of that used in malignancies. The typical starting dose is 0.5 to 1.5 mg/kg/day 6-mercaptopurine with a usual maximum of ≦50 mg/day (Bernstein, et al. (1994) Dig. Dis. Sci. 39(8):1638-41). At this dosage, minimal hematological toxicity is caused.

Similarly, methotrexate is used in “high” and “low” doses for treatment of different diseases. For example, the high dose is used for malignancies such as non-Hodgkin's lymphoma (Urba, et al. (1990) J. Natl. Cancer Inst. Monogr. 10:29-37). In combination with other anti-neoplastic agents, the dose of methotrexate is 120 mg/M² over 8 days as an oral dosage, and 1500-5000 mg M² by IV infusion over 2-24 hours (Findley & Fortner (1996) In: Herfindal E T, Gourley D R, eds. Textbook of Therapeutics: Drugs and Disease Management. Sixth ed. Baltimore Md.: Williams & Wilkins, pg. 1509-1513).

In contrast, “low” dose methotrexate is used for inflammatory diseases such as such as IBD, rheumatoid arthritis and psoriasis. In IBD, (Crohn's and ulcerative colitis) a weekly injection of 25 mg improves symptoms and decreases the required dose of corticosteroids (Feagan, et al. (1995) N. Engl. J. Med. 332(5):292-7). In the treatment of rheumatoid arthritis the dose of methotrexate is 7.5 to 15 mg orally or by IM injection weekly (Weinblatt, et al. (1992) Arthritis Rheum. 35(2):129-37; Weinblatt, et al. (1998) J. Rheumatol. 25(2):238-42). Similarly, in the therapy of psoriasis, the dose of methotrexate is 5 mg to 7.5 mg weekly.

The effectiveness of these “low” dose 6-mercaptopurine and methotrexate regimes in the treatment of inflammatory diseases is herein posited to be as a result of antibiotic activity which decreases bacterial infectious burden, with a decrease in pro-inflammatory cytokines as a secondary phenomenon.

Similarly, there are disparate doses of thalidomide that are used to treat malignant, infectious and “inflammatory” diseases. When used to treat malignancies, (such as the reticuloendothelial malignancy Multiple Myeloma) the initial dose of thalidomide is 200 mg/day increasing to 1200 mg/day (Singhal, et al. (1999) N. Engl. J. Med. 341(21):1565-71). In contrast, a lower dose is used to treat the acknowledged infectious disease erythema nodosum leprosum (ENL,) that is caused by M. leprae. The initial dose of thalidomide for ENL is 200 mg/day. It is increased to 400 mg/day, but the maintenance dose is 50 mg/day to 200 mg/day (Iyer, et al. (1971) Bull. World Health Organ. 45(6):719-32). Thus, the maintenance dose of thalidomide for a mycobacterial infectious disease may be one sixteenth of that used for reticuloendothelial malignancies. The dose of thalidomide that is used in the therapy of Crohn's disease (herein posited to be primarily infectious and due to M. paratuberculosis rather than primarily “inflammatory”) is similar to that used in ENL the other mycobacterial disease discussed, and is one-eighth the dose used to treat reticuloendothelial malignancies. In refractory Crohn's disease, the starting dose of thalidomide is 200 mg/day, decreasing to maintenance of 100 mg/day to 200 mg/day (Ehrenpreis, et al. (1999) Gastroenterology 117(6):1271-7).

Likewise, “high” and “low” doses of immunosuppressants are used depending on whether the immunosuppressant is being administered to reduce organ transplant rejection or in the treatment of “inflammatory” diseases. For example, the targeted trough level of cyclosporine A in transplant rejection is 100 ng/ml to 400 ng/ml (Wong (2001) Clin. Chim. Acta 313(1-2):241-53; van der Pijl (1996) Transplantation; 62(4):456-62; Lindholm & Kahan (1993) Clin. Pharmacol. Ther. 54(2):205-18), whereas levels in the range of 70 ng/ml to 200 ng/ml are recommended in the treatment of Crohn's disease. “High” dose targeted trough levels of tacrolimus, in the range of 5 ng/mL to 20 ng/mL (Wong (2001) supra), are used, e.g., in liver transplantation (Sugawara, et al. (2001) Clin. Transplant. 16(2):102-6), whereas “low” targeted trough levels of 4-8 ng/ml, or 5-10 ng/ml are recommended in the treatment of inflammatory bowel disease (Baumgart, et al. (2006) Am. J. Gastroenterol. 101(5):1048-56; de Oca, et al. (2003) Rev. Esp. Enferm. Dig. 95(7):465-70, 459-64).

Accordingly, an effective amount of each anti-MAP agent can be routinely determined by the skilled physician, in the light of the teachings herein and the relevant circumstances, including the condition to be treated, the chosen route of administration, the actual compound administered, the age, weight, and the like.

Using the methods of this invention, prevention or treatment of a MAP infection is achieved. In so far as the MAP infection being prevented or treated is associated with, e.g., an inflammatory bowel disease (IBD), Multiple Sclerosis or Alzheimer's Disease, prevention or treatment will also delay, eliminate, improve or ameliorate at least one sign or symptom of such diseases or the provide maintenance of disease remission.

The invention is described in greater detail by the following non-limiting examples.

### Example 1

### Anti-Mycobacterial Vaccine

Mice (10 per group), e.g., wild-type and/or IL-18 deficient mice (Momotani, et al. (2002) Proc. 7^{th} Intl. Coll. Paratuberculosis, Juste (ed)) are immunized intraperitoneally (i.p.) with either AhpC or AhpD protein (15 μg in 50 μl PBS (phosphate-buffered saline) in combination with an Ag85B-ESAT-6 fusion protein (15 μg in 50 μl PBS) emulsified in 50 μl complete Freund's adjuvant (CFA)). A group of 10 mice are sham-immunized with PBS and CFA only.

A second immunization of 15 μg of each antigen with incomplete Freund's adjuvant (IFA) is administered 3 weeks later (with the sham-immunized group receiving PBS and IFA).

Blood is drawn at weeks 5 and 7. Sera from each group are pooled for analysis of antigen-specific antibody production by ELISA. Mice are challenged at week 8 by intraperitoneal injection of MAP and M. tuberculosis. Mice are monitored for signs and symptoms of disease.

Data will indicate that immunization of mice with either recombinant AhpC or AhpD proteins in combination with the Ag85B-ESAT-6 fusion protein elicits a response capable of protecting against MAP and M. tuberculosis infection.

### Example 2

### Immunogenicity of Anti-Mycobacterial Vaccine in Humans

Sera from patients with culture-proven MAP infection are used in western blot analysis containing recombinant AhpC or AhpD protein.

The results of this analysis will demonstrate that sera from patients with MAP infections exhibit reactivity with either AhpC or AhpD, thereby indicating that AhpC and AhpD are recognized by the human immune system and suggest that antibodies able to bind the AhpC or AhpD protein can be produced during natural MAP infection in humans. Further, this data provides evidence that AhpC and AhpD are expressed in vivo by MAP during infection, and thus can be available as targets for immunoprophylaxis, immunotherapy, or to provide immune responses in subjects vaccinated with these proteins.

### Example 3

### Methotrexate and 6-MP Inhibition of MAP Proliferation

Bacterial quantification was performed retrospectively. Accordingly, for experimental reproducibility, bacterial passage and harvesting were performed when the GI was ˜500. Quantification show that the CFU's of the M. avium isolates were approximately 10-fold higher (˜1×10⁷ CFUs/ml), compared to MAP (˜1×10⁶ CFUs/ml) (Table 2). Because of the difference in growth kinetics, M. avium CFU numbers inoculated were ≧10-fold lower than MAP (Table 2).

TABLE 2

| | MAP | | | M. avium | |
|---|---|---|---|---|---|
| | ATCC | ATCC | | ATCC | |
| | 19698 | 19698 | Dominic | 25291 | 101 |
| GI at | 526 | 523 | 548 | 669 | 267 |
| harvest | | | | | |
| Harvested | 8.1 × 10⁵ | 8.2 × 10⁵ | 6.3 × 10⁵ | 9.1 × 10⁶ | 1.2 × 10⁶ |
| # CFU's/ml | | | | | |
| # CFU's | 20,250 | 20,500 | 15,750 | 910 | 120 |
| Inoculated | | | | | |
| Days to | 12 | 13 | 17 | 7 | 5 |
| reach GI | | | | | |
| “999” | | | | | |

Both MAP isolates (ATCC 19698 & Dominic) were Mycobactin J-dependant, were IS 900-positive and had ≧99% homology with the GENBANK accession NC_{—}002944 of MAP. M. avium ATCC 25291 was positive for IS 902 and M. avium 101 was negative for both.

In this study, the positive control antibiotic, clarithromycin exhibited ≧86%−ΔcGI at the lowest concentration evaluated (0.5 μg/ml). The negative control antibiotic (the β lactam, ampicillin) had a minimal effect (21%−ΔcGI) at the 32 μg/ml. In contrast, 6-MP had an initial ≧43%−ΔcGI starting at 1 μg/ml increasing to ≧84%−ΔcGI at 4 μg/ml, whereas methotrexate had 40%−ΔcGI inhibition at 2 μg/ml and ≧75%−ΔcGI at 4 μg/ml.

The effect of methotrexate and 6-MP against two MAP and two M. avium isolates was also evaluated. In these studies, the MAP 19698 results showed ˜80%−ΔcGI inhibition at 4 μg/ml for both 6-MP and clarithromycin. In contrast, MAP Dominic showed less susceptibility to 6-MP (41%−ΔcGI at 4 μg/ml) compared to MAP 19698 (84%−ΔcGI at 4 μg/ml). Both M. avium isolates showed less susceptibility to 6-MP than to methotrexate. The diluent control inoculum for the M. avium ATCC 25291 appeared to exhibit complete growth inhibition. However, over the following two days this methanol control entered log phase growth, whereas the vials at every clarithromycin dose continued to show no evidence of growth.

The results of this analysis indicate that both methotrexate and 6-MP inhibit the growth kinetics of MAP. As is conventional with antibiotic susceptibility studies, the present analysis compared agents on an equal weight basis. However, methotrexate (MW 450) is a much larger molecule than 6-MP (MW 170) with a molar ratio of ˜3:1. Thus, in comparison to 6-MP on a molar basis, methotrexate is an even more potent inhibitor of growth than the present data indicate. Moreover, it is expected that combinations of anti-MAP agents will exhibit dose-dependent, growth suppressive activity against MAP which surpass the inhibitory activity of each agent alone. Accordingly, the present invention embraces compositions and methods for inhibiting the proliferation of MAP and treating or ameliorating a MAP infection. Specifically, the present invention provides compositions containing one or more anti-MAP agents including a folate antagonist, a purine inhibitor, a thymidylate synthase inhibitor, an antibiotic, a salicylic acid and a thalidomide, as anti-mycobacterial agents for suppressing the growth of MAP thereby treating diseases or conditions associated with MAP (i.e., MAP is the etiological agent of the disease or condition).

### Example 4

### Inhibition of MAP Proliferation with Salicylic Acids and a Sulfonamide

To demonstrate the efficacy of salicyclic acids and a sulfonamide folate antagonist in the inhibition of MAP proliferation, 5-ASA, p-aminosalicylic acid, and sulfapyridine were evaluated as individual compounds. Moreover, combinations of agents were tested as was the 5-ASA and sulfapyridine conjugate, sulfasalazine. This analysis indicated that 5-ASA and p-aminosalicylic acid inhibited the proliferation of MAP (ATCC 19698) in a dose-dependent manner, whereas sulfasalazine and sulfapyridine had little effect on the cumulative growth index of MAP. On the other hand, sulfapyridine and 5-ASA+sulfapyridine inhibited M. avium 101 in a dose-dependent manner, whereas 5-ASA had little effect.

Accordingly, these data demonstrate that there may be differences in the response of individual MAP isolates (e.g., isolated from different patients) to anti-MAP agents. For example, it is expected that MAP strains, ATCC 43015, ATCC 43544, ATCC 43545, ATCC 49164, ATCC 70053, K10 and JTC303, as well as clinical isolates from both veterinary and human sources, will respond differently to different individual compounds and combinations of compounds (sulfapyridine and 5-ASA). Therefore, as disclosed herein, it will be essential to determine the antibiotic susceptibility of a MAP isolated from a subject in order to select an appropriate anti-MAP agent, or combination of anti-MAP agents, to effectively treat a MAP infection.

### Example 5

### Inhibition of MAP Proliferation with Thalidomide

Racemic thalidomide (±), as well as the (+) and (−) enantiomers, were analyzed for anti-MAP activity. In addition, MAP growth kinetics were determined in the presence of phthalimide and 1-Hydroxy-2,6-piperidinedione (HPD). This analysis was carried out using a human (Dominic) and bovine (ATCC 19698) strain of MAP and two strains of M. avium (ATCC 25291 and 101.) A radiometric (¹⁴CO₂ BACTEC®) detection system was used to quantify mycobacterial growth as arbitrary “growth index units” (GI.) Efficacy data are presented as “percent decrease in cumulative GI” (%−ΔcGI.)

The results of this analysis indicated that phthalimide did not exhibit a dose-dependent inhibition of any strain. Furthermore, there was no dose-dependent inhibition on either M. avium subspecies avium strain with thalidomide (Table 3), phthalimide or HPD. In contrast, dose-dependent inhibition was observed for thalidomide (±) (31%−ΔcGI for Dominic and 26%−ΔcGI for ATCC 19698 at 64 μg/ml thalidomide; Table 3), with thalidomide (+) (Table 4) being more inhibitory than thalidomide (−). HPD was, on a weight-for-weight basis, the most potent inhibitory agent evaluated (46%−ΔcGI for Dominic and −44%−ΔcGI for ATCC 19698 at 64 μg/ml HPD; Table 5).

TABLE 3

| | %-ΔcGI | | | | |
|---|---|---|---|---|---|
| | MAP | | | | |
| Thalidomide | Dominic | | | M. avium | |
| (±) (μg/ml) | Expt A | Expt B | ATCC 19698 | ATCC 25291 | 101 |
| 1 | −2% | 0% | 4% | 34% | −20% |
| 4 | −4% | −2% | 8% | 37% | −5% |
| 16 | −9% | −5% | −7% | 28% | 6% |
| 64 | −31% | −13% | −26% | 25% | −14% |

TABLE 4

| | |
|---|---|
| Thalidomide | %-ΔcGI |

| (+) (μg/ml) | MAP (Dominic) | M. avium (101) |
|---|---|---|
| 1 | −4% | 4% |
| 4 | 1% | −5% |
| 16 | −14% | 16% |
| 64 | −58% | −19% |

TABLE 5

| | %-ΔcGI | | | | |
|---|---|---|---|---|---|
| | MAP | | | | |
| HPD | Dominic | | | M. avium | |
| (μg/ml) | Expt A | Expt B | ATCC 19698 | ATCC 25291 | 101 |
| 1 | −5% | 16% | 22% | −4% | −12% |
| 4 | −7% | 6% | 7% | 47% | −52% |
| 16 | −18% | 12% | −3% | 22% | −21% |
| 64 | −46% | −19% | −44% | 21% | −36% |

### Example 6

### Inhibition of MAP Proliferation with Immunosuppressants

Known immunosuppressive agents were also analyzed for anti-MAP activity. Specifically, Cyclosporine A, Tacrolimus (FK506) and Rapamycin were analyzed for anti-MAP activity against both human and bovine strains of MAP, M. avium subspecies avium and BCG (as a level II surrogate for M. tuberculosis). Six strains of mycobacteria were analyzed, four of which were MAP. Two MAP strains were isolated from humans with Crohn's disease: Dominic (ATCC 43545; Chiodini, et al. (1986) J. Clin. Microbiol. 24:357-363) and UCF 4 (Naser, et al. (2004) Lancet 364:1039-1044). The other two MAP strains were from ruminants with Johne's disease ATCC 19698 (ATCC, Rockville, Md.) and 303 (Sung & Collins (2003) Appl. Environ. Microbiol. 69(11):6833-6840). The M. avium subspecies avium strains (hereinafter called M. avium) were ATCC 25291 (veterinary source) and M. avium 101 (Betram, et al. (1986) J. Infect. Dis. 154:194-195).

Because it renders clinically resistant strains of MAP inappropriately susceptible to antimicrobials in cell culture (Damato & Collins (1990) Vet. Microbiol. 22:31-42), TWEEN 80 detergent was not included in the cultures. Prior to inoculation, cultures were processed as described herein and according to conventional methods (Rastogi, et al. (1992) Antimicrobiol. Agents Chemother. 36:2843-2846).

For experimental comparability, only chemicals that could be solubilized with DMSO were used. The positive control antibiotics were clofazimine, an antibiotic used to treat leprosy (Britton & Lockwood (2004) Lancet 363:1209-1219) and in clinical trials for the treatment of Crohn's disease (Selby, et al. (2007) Gastroenterology 132:2313-2319; Borody, et al. (2002) Dig. Liver Dis. 34:29-38), and monensin, an antibiotic used in veterinarian medicine (McDougald (1976) Poult. Sci. 55:2442-2447; Melendez, et al. (2006) Prev. Vet. Med. 73:33-42). The two negative controls were gluterimide antibiotics, cycloheximide and phthalimide.

The tested agents, Cyclosporine A, Rapamycin and Tacrolimus (all Sigma, St. Louis, Mo.) were solubilized in 100% DMSO. Aliquots were prediluted, stored at −80° C. in 50% DMSO (Sigma) in water, thawed, used once and discarded. Volumes of DMSO were adjusted so that final concentration in every BACTEC® vial used was always 3.2% DMSO. Agents were tested in serial dilutions from a minimum of 0.5 μg/ml to a maximum of 64 μg/ml. Inhibition of mycobacterial growth is expressed as %−ΔcGI, and enhancement as %+ΔcGI compared to 3.2% DMSO controls. In other words, a negative % ΔcGI is the percent decrease in cumulative GI compared to control inoculation.

Data for individual chemical agents are presented in Tables 6-12. The positive experimental controls were clofazimine (Table 6) and monensin (Table 7). The “negative” controls were cycloheximide (Table 8) and phthalimide (Table 9). Tables 10-12 present data for Cyclosporine A, Rapamycin, and Tacrolimus, respectively.

TABLE 6

| | Mycobacterial strain (% ΔcGI) | | | | | | |
|---|---|---|---|---|---|---|---|
| | M. avium subspecies paratuberculosis | | | | | | |
| | Human MAP | | | | | M. avium | |
| Clofazimine | Dominic | Dominic | | Bovine MAP | | Bovine | |
| (μg/ml) | Expt. 1 | Expt. 2 | UCF4 | 303 | 19698 | 25291 | 101 |
| 1 | −99 | −99 | −99 | −99 | −99 | −98 | −98 |
| 4 | −99 | −99 | −99 | −99 | −99 | −98 | −98 |
| 16 | −99 | −99 | −99 | −99 | −99 | −98 | −98 |
| 64 | −99 | −99 | −99 | −99 | −99 | −99 | −99 |

TABLE 7

| | Mycobacterial strain (% ΔcGI) | | | | | | |
|---|---|---|---|---|---|---|---|
| | M. avium subspecies paratuberculosis | | | | | | |
| | Human MAP | | | | | M. avium | |
| Monensin | Dominic | Dominic | | Bovine MAP | | Bovine | |
| (μg/ml) | Expt. 1 | Expt. 2 | UCF4 | 303 | 19698 | 25291 | 101 |
| 1 | −52 | −46 | −73 | −21 | −14 | −25 | 7 |
| 4 | −77 | −71 | −93 | −73 | −45 | −54 | 3 |
| 16 | −93 | −87 | −96 | −94 | −62 | −69 | 4 |
| 64 | −94 | −92 | −97 | −97 | −65 | −87 | 8 |

TABLE 8

| | Mycobacterial strain (% ΔcGI) | | | | | | |
|---|---|---|---|---|---|---|---|
| | M. avium subspecies paratuberculosis | | | | | | |
| | Human MAP | | | | | M. avium | |
| Cycloheximide | Dominic | Dominic | | Bovine MAP | | Bovine | |
| (μg/ml) | Expt. 1 | Expt. 2 | UCF4 | 303 | 19698 | 25291 | 101 |
| 1 | 15 | 9 | 1 | 3 | −2 | 0 | 6 |
| 4 | −4 | 0 | −7 | 4 | −7 | −8 | 7 |
| 16 | −8 | −4 | −4 | −5 | −12 | −7 | 1 |
| 64 | −1 | −7 | −12 | −4 | −9 | −57 | 5 |

TABLE 9

| | Mycobacterial strain (% ΔcGI) | | | | | | |
|---|---|---|---|---|---|---|---|
| | M. avium subspecies paratuberculosis | | | | | | |
| | Human MAP | | | | | M. avium | |
| Phthalimide | Dominic | Dominic | | Bovine MAP | | Bovine | |
| (μg/ml) | Expt. 1 | Expt. 2 | UCF4 | 303 | 19698 | 25291 | 101 |
| 1 | −3 | −1 | 4 | −2 | −2 | 2 | −3 |
| 4 | 7 | 0 | −2 | 3 | −10 | 4 | 4 |
| 16 | 0 | 1 | 1 | −13 | 1 | 1 | 2 |
| 64 | 2 | 4 | 3 | 6 | 0 | 12 | −2 |

TABLE 10

| | Mycobacterial strain (% ΔcGI) | | | | | | |
|---|---|---|---|---|---|---|---|
| | M. avium subspecies paratuberculosis | | | | | | |
| | Human MAP | | | | | M. avium | |
| Cyclosporin | Dominic | Dominic | | Bovine MAP | | Bovine | |
| A (μg/ml) | Expt. 1 | Expt. 2 | UCF4 | 303 | 19698 | 25291 | 101 |
| 1 | 15 | 4 | −2 | −2 | −2 | 22 | −28 |
| 4 | −10 | −9 | 5 | −3 | −23 | 31 | −44 |
| 16 | −43 | −64 | −9 | −14 | −19 | 4 | −56 |
| 64 | −98 | −99 | −99 | −91 | −92 | −54 | −95 |

TABLE 11

| | Mycobacterial strain (% ΔcGI) | | | | | | |
|---|---|---|---|---|---|---|---|
| | M. avium subspecies paratuberculosis | | | | | | |
| | Human MAP | | | | | M. avium | |
| Rapamycin | Dominic | Dominic | | Bovine MAP | | Bovine | |
| (μg/ml) | Expt. 1 | Expt. 2 | UCF4 | 303 | 19698 | 25291 | 101 |
| 1 | 21 | 0 | −14 | 4 | −3 | 10 | −19 |
| 4 | 13 | −7 | −9 | −11 | −1 | 7 | −7 |
| 16 | −10 | −9 | −29 | −15 | −18 | 11 | −28 |
| 64 | −58 | −44 | −76 | −39 | −43 | −18 | −39 |

TABLE 12

| | Mycobacterial strain (% ΔcGI) | | | | | | |
|---|---|---|---|---|---|---|---|
| | M. avium subspecies paratuberculosis | | | | | | |
| | Human MAP | | | | | M. avium | |
| Tacrolimus | Dominic | Dominic | | Bovine MAP | | Bovine | |
| (μg/ml) | Expt. 1 | Expt. 2 | UCF4 | 303 | 19698 | 25291 | 101 |
| 1 | 28 | 5 | 6 | 5 | 8 | −23 | −34 |
| 4 | 9 | 9 | −19 | 3 | −3 | −3 | −43 |
| 16 | 21 | −10 | −18 | −5 | −5 | 11 | −40 |
| 64 | 0 | −21 | −43 | −27 | −26 | 53 | −52 |

The most potent positive control was clofazimine, 97%−ΔcGI at 0.5 (Dominic; Table 6). The other positive control, Monensin, had dose-dependent inhibition against all MAP strains. It was most effective against MAP isolated from humans (Table 7) and bovine 303, and least against bovine ATCC 25291 (Table 7). Monensin had no inhibition against M. avium 101 (Table 7).

The negative control chemical agents were the gluterimide antibiotics cycloheximide and phthalimide. Cycloheximide had no dose-dependent inhibition on any MAP strain (Table 8). Cycloheximide had dose-dependent inhibition on M. avium ATCC 25291, (57%−ΔcGI at 64 μg/ml) but no affect on M. avium 101 (Table 8). Phthalimide had no dose-dependent effect on any strain tested (Table 9).

The three “immunosuppressants” tested were Cyclosporine A, Rapamycin and Tacrolimus. There were differing amounts of inhibition depending on the agent and strain. The control mycobacterial strains were M. avium subspecies avium ATCC 25291 and 101. Of the three “immunosuppressants,” Cyclosporine A had dose-dependent inhibition on M. avium subspecies avium 101 (95%−ΔcGI at μg/ml) (Table 10). There was no inhibition with Rapamycin or Tacrolimus on the control M. avium 25291 (Tables 11 and 12).

Against MAP, Cyclosporine A was the most effective of the three “immunosuppressants” studied. On MAP isolated from humans, (Dominic and UCF 4), Cyclosporine had 97%−ΔcGI at 32 μg/ml against Dominic and 99%−ΔcGI at 64 μg/ml on Dominic and UCF 4 (Table 10). On MAP isolated from ruminants, Cyclosporine A had slightly less dose-dependent inhibition (ATCC 19698: 92%−ΔcGI at 64 μg/ml) than against MAP isolated from humans (Table 10). Rapamycin was the second most effective “immunosuppressant” studied. At lower concentrations (1 and 16 μg/ml) Rapamycin had no inhibition and at 64 μg/ml it had 76%−ΔcGI on UCF 4, a MAP isolated from humans (Table 11). Rapamycin was less effective against MAP isolated from ruminants and had no effect on M. avium ATCC 25291 (Table 11). Tacrolimus had the least inhibition of the three “immunosuppressants” studied. Against MAP, Tacrolimus was most inhibitory against UCF 4 (43%−ΔcGI at 64 μg/ml) and ATCC 19698 (26%−ΔcGI at 64 μg/ml) (Table 12). Paradoxically, Tacrolimus exhibited the most inhibition on M. avium 101 of all six strains studied, yet actually enhanced growth of M. avium ATCC 25291 (Table 12).

Rapamycin was initially evaluated as an anti-fungal agent (Singh, et al. (1979) J. Antibiot. (Tokyo) 32:630-654). However, this is the first time that anti-MAP activity has been demonstrated for the “immunosuppressant” agents Cyclosporine A, Rapamycin and Tacrolimus. These observations therefore further demonstrate MAP is responsible for multiple “autoimmune” and “inflammatory” diseases, and that the action of these three “immunosuppressant” agents may simply be to inhibit MAP growth.

As with the “high” and “low” doses of methotrexate and 6-MP used in the treatment of human malignancies and “autoimmune” or “inflammatory” conditions, respectively, there are “high” and “low” doses of the three “immunosuppressants” studied herein, wherein “low” doses have prokaryotic antibiotic action in addition to eukaryotic immunosuppressant activity. The data presented herein are subtle and the negative controls are critical. It must be emphasized that these data were obtained using the exquisitely sensitive radiometric ¹⁴C Bactec® System. As with 5-ASA, the effects of Cyclosporine A, Rapamycin and Tacrolimus may hot be detectable using the more convenient, fluorescent-based MIGT System®.

What is claimed is:

1. A method for preventing or treating a disease or condition associated with a Mycobacterium avium subspecies paratuberculosis (MAP) infection comprising
(a) screening a subject for the presence of one or more markers indicative of predisposition or susceptibility to a disease or condition associated with a MAP infection;
(b) selecting a vaccine or prophylactic or therapeutic agent for the subject based upon the presence of the one or more markers; and
(c) administering to the subject the vaccine or prophylactic or therapeutic agent to prevent or treat the disease or condition associated with the MAP infection.

2. The method of claim 1, wherein the vaccine comprises at least one Mycobacterium avium subspecies paratuberculosis (MAP) antigen, or attenuated or killed MAP.

3. The method of claim 2, wherein the attenuated or killed MAP is cell wall-competent or cell wall-deficient.

4. The method of claim 2, wherein the MAP antigen is GroES, AhpD, 32 kDa antigen, 34 kDa antigen, 34.5 kDa antigen, 35 kDa antigen, 36 kDa antigen, 42 kDa antigen, 44.3 kDa antigen, 65 kDa antigen or AhpC antigen.

5. The method of claim 2, wherein the vaccine further comprises at least one antigen isolated from a member of the M. tuberculosis complex (MTC), or an attenuated or killed mycobacterium from the MTC.

6. The method of claim 5, wherein the member of the MTC is selected from the group of M. tuberculosis, M. bovis, M. bovis Calmette-Guérin, M. africanum, M. canetti, M. caprae, M. pinnipedii and M. microti.

7. The method of claim 1, wherein the prophylactic or therapeutic agent comprises one or more of a folate antagonist, a salicylic acid, a purine inhibitor, a thymidylate synthase inhibitor, an antibiotic, an immunosuppressant, a thalidomide, or a prodrug or metabolite thereof.
